(19) United States
(12) Plant Patent Application Publication
(10) Pub. No.: US 2002/0040491 P1
(43) Pub. Date: Apr. 4, 2002

(54) Chrysanthemum plant named 'Redock Salmon'

(76) Inventor: Robert Noodelijk, Woubrugge (NL)

Correspondence Address: PARKHURST & WENDEL, SUITE 210, 1421 PRINCE STREET, ALEXANDRIA, VA, 22314, US

(21) Appl. No.: 09/910,027

(22) Filed: Jul. 23, 2001

(30) Foreign Application Priority Data
Jul. 25, 2000 (NL) CHR3210
Jul. 25, 2000 (EP) 0963/2001

(51) Int. Cl. A01H 5/00

(52) U.S. Cl. PLT/286

(57) ABSTRACT

A Chrysanthemum plant named ‘Redock Salmon’ characterized by its medium sized blooms with salmon ray-florets and yellow-green disc florets.

### RELATED CULTIVARS

‘Redock Salmon’ is related to ‘Redock Dark (#................) and ‘Redock Orange’ (#.................). These varieties are all mutants of the original ‘Redock’ (unpatented).

### BACKGROUND OF THE INVENTION

‘Redock Salmon’ is a product of a breeding and selection program which had the objective of finding color mutants of ‘Redock’. The new plant of the present invention comprises a new and distinct cultivar of chrysanthemum plant that is a natural occuring sport of a parent chrysanthemum named ‘Redock’ (unpatented). A comparison with Parent chrysanthemum ‘Redock’ is also made in this application. The new cultivar was discovered as a whole plant mutation in September 1999 by Rob Noodelijk in a controlled environment (greenhouse) in Rijsenhout Holland. The first act of asexual reproduction of ‘Redock Salmon’ was accomplished when vegetative cuttings were taken from the initial selection in November 1999 in Rijsenhout Holland.

### SUMMARY OF THE INVENTION

The present invention is a new and distinct variety of chrysanthemum bearing medium sized blooms with salmon ray-florets and yellow-green disc florets.

### BRIEF DESCRIPTION OF THE DRAWINGS

The present invention of a new and distinct variety of chrysanthemum is shown in the accompanying drawings, the color being as nearly true as possible with color photographs of this type.

FIG. 1 shows a plant of the cultivar in full bloom.

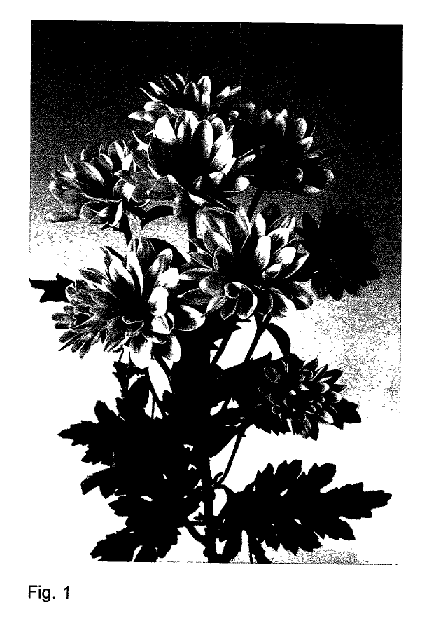

FIG. 2 shows the foliage of the cultivar.

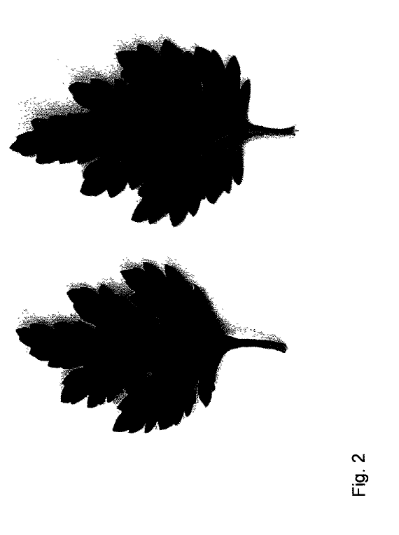

### DESCRIPTION OF THE INVENTION

This new variety of chrysanthemum is of the botanical classification Dendranthema grandiflora. The observations and measurements were gathered from plants grown in April/May in a greenhouse in Rijsenhout Holland in a photo-periodic controlled crop under conditions generally used in commercial practice. The greenhouse temperatures during this crop were at day-time between 18° C. and 25° C. and at night 20° C. After a long day period of 14 days the photo-periodic response time in this crop was 47 days. After the long day period to flowering growth retardants were applied 3 times in an average dose of 2.5 gram/liter water. No tests were done on disease or insect resistance or susceptibility. This new variety produces medium sized blooms with salmon ray-florets and yellow-green disc-florets blooming on the plant for 4 weeks. This new variety of chrysanthemum has been found to retain its distinctive characteristics throughout successive propagations however the phenotype may vary significantly with variations in environment such as light intensity and temperature. To show the phenotype as described ‘Redock Salmon’ can be planted without assimilation lightning (high pressure sodium lamps) between week 50 and week 40 of the next year under greenhouse conditions in Holland. With assimilation light (minimum level 2500 lux) it can be planted year round under greenhouse conditions in Holland.

From the cultivars known to inventor the most similar existing cultivar in comparison to ‘Redock Salmon’ is ‘Redock Orange’. When ‘Redock Salmon’ and ‘Redock Orange’ are being compared the following differences and similarities are noticed: The difference of ‘Redock Orange’ and ‘Redock Salmon’ is the ray-floret color. All other characteristics of ‘Redock Orange’ and ‘Redock Salmon’ are similar.

From other commercial varieties ‘Salmon Reagan’ (USPP 8769) and ‘Salmon Elite Reagan’ (not patented yet) are the most similar. The differences between ‘Salmon Reagan’, ‘Salmon Elite Reagan’ and ‘Redock Salmon’ are (1) Flower form and color. The ray-florets of ‘Redock Salmon’ are more upright, less broad, more pointed and more concave. (2) Vigour. Under the same circumstances the plants of ‘Redock Salmon’ are 20 cm longer. (3) Response time. The response time of ‘Redock Salmon’ is shorter. (4) The disc-floret size of ‘Redock Salmon’ is smaller. (5) Foliage size and color. The foliage of ‘Redock Salmon’ is bigger and more yellow-green.

The following is a description of the plant and characteristics that distinguish ‘Redock Salmon’ as a new and distinct variety. The color designations are taken from the plant itself. Accordingly, any discrepancies between the color designations and the colors depicted in the photographs are due to photographic tolerances. The color chart used in this description is: The Royal Horticultural Society color chart, edition 1995.

TABLE 1
Botanical Description of CULTIVAR ’Redock Salmon’

| | |
|---|---|
| Bud | |
| Size | Small, cross-section |
| | +/−0.7 cm height +/−0.9 cm |
| Outside Color | Orange 27 A |
| Involucral bracts | 3 rows, length 8 mm, width 3 mm |
| Involucral bracts among disc-florets | Not present |
| Involucral bracts color | Green 147 B |
| Bloom | |
| Type | Single |
| Height | High |
| Size | Medium |
| Fully Expanded | 7.0 cm |
| Number of blooms per single stem | Average of 14 |
| Seeds | Not produced |
| Performance (blooming period) | 4 weeks |
| Fragrance | Typical chrysanthemum |
| Color | |
| Center of the flower (disc-florets) | Immature yellow-green 144 A |
| | Mature yellow-green 151 C |
| Color of upper surface of the | Orange 29 D |
| majority of the ray-florets | |
| Color of the lower surface of the | Yellow-orange 19 D |
| majority of the ray-florets | |
| Tonality from Distance | A spray mum with pink flowers |
| | and a yellow-green disc |
| Discoloration to color | To color yellow-orange 19 D |
| Ray florets | |
| Texture | Upper and under side smooth |
| Number | 26-28 |
| Cross-section | Strongly concave |
| Longitudinal axis of majority | Reflexing |
| Length of corolla tube | Short |
| Ray-floret length | 3.3 cm |
| Ray-floret width | 1.0 cm |
| Ratio length/width | Medium |
| Shape of tip | Pointed, very occasionally round |
| Disc florets | |
| Disc diameter | 0.8 -1.0 cm |
| Distribution of disc florets | Numerous, clearly visible at all |
| | stages of flowering |
| Shape | Tubular |
| Color | Yellow-green 144 A |
| Receptacle shape | Conical raised |
| Reproductive Organs | |
| Stamen (present in disc florets only) | Thin 3 mm in length |
| Stamen color | Yellow-green 144 B |
| Pollen | No pollen present |
| Pollen color | Not applicable |
| Styles (present in both ray and disc | Thin |
| florets) | |
| Style color | Yellow-green 144 B |
| Style Length | 4 mm |
| Stigmas | Yellow-green 144 B |
| Stigma Width | 2 mm |
| Ovaries | Enclosed in calyx |
| Plant | |
| Form | A spray mum meant for erect |
| | culture |
| Growth habit | Upright |
| Growth rate | Very vigorous |
| Height | 125-135 cm |
| Internodes | 2.5-3.0 cm |
| Spray formation | Corymbiform |
| Stem Color | Yellow-green 147 B |
| Stem Strength | Medium |
| Stem Brittleness | Present |
| Stem Anthocyanin Coloration | Absent |
| Peduncle length | Near the top 16 cm, near the |
| | middle 23 cm, near the bottom |
| | 28 cm |
| Peduncle color | Green 147 B |
| Peduncle, attachment | Brittle |
| Peduncle, angle with stem | Small, 30° C. |
| Flowering Response (photo-periodic | 47 days |
| controlled crop, not natural season) | |
| Foliage | |
| Color | Upper side yellow-green 147 A |
| | Under side green 138 B |
| Size | large; length 12.0-14.0 cm, |
| | width 7.0-8.0 cm |
| Quantity (number per single stem) | 30-34 |
| Shape | Ovate and deeply lobed |
| Texture upper side | Fleshy and glabrous |
| Texture under side | Pubescent |
| Ribs and veins upper side | Ribs and veins well developed |
| Ribs and veins under side | Ribs and veins well developed |
| Venation arrangement | Palmate |
| Shape of the margin | Lobed |
| Shape of Base of Sinus Between | Round |
| Lateral Lobes | |
| Margin of Sinus Between Lateral | Converging |
| Lobes | |
| Shape of Base | Rounded |
| Apex | Mucronate |

TABLE 2
Differences with the comparison Varieties

| | | | | ’Salmon |
|---|---|---|---|---|
| | ’Redock | ’Redock | ’Salmon | Elite |
| | Salmon' | Orange' | Reagan' | Reagan' |
| Ray-floret | Orange 29 D | Orange 26 B | Orange 29 C | Yellow- |
| color | | | | Orange 19B |
| Flower height | High | High | Flat | Flat |
| Cross-section | Strongly | Strongly | flat | Flat |
| of ray-floret | concave | concave | | |
| Vigour | Very | Very | Vigorous | Vigorous |
| | vigorous | vigorous | | |
| Plant height | 125-135 cm | 125-135 cm | 100-125 cm | 105-130 cm |

I claim:

1. A new and distinct variety of chrysanthemum plant as described and illustrated.
